(19) United States
(12) Patent Application Publication
(10) Pub. No.: US 2015/0018707 A1
(43) Pub. Date: Jan. 15, 2015

(54) PAIN ANALYSIS USING ELECTRODERMAL ACTIVITY

(71) Applicant: Affectiva, Inc., Waltham, MA (US)

(72) Inventors: Viprali Bhatkar, Cambridge, MA (US); Evan Kodra, Cambridge, MA (US); Rosalind Wright Picard, Newtonville, MA (US); Camilla Staahl, Skoerping (DK)

(21) Appl. No.: 14/329,013

(22) Filed: Jul. 11, 2014

Related U.S. Application Data
(60) Provisional application No. 61/845,932, filed on Jul. 12, 2013.

(51) Int. Cl. A61B 5/00; A61B 5/053

(52) U.S. Cl. CPC A61B 5/7278; A61B 5/0533; A61B 5/4824; A61B 5/725; A61B 5/4848
USPC 600/547

(57) ABSTRACT

A method for accurate pain analysis using a combination of electrodermal data and self-report information is described. To obtain pain measurements, electrodermal activity is collected and analyzed. One or more biosensors obtain electrodermal data from a person. The electrodermal data is filtered to produce skin conductance values. The skin conductance values are associated with self-report pain information from a person over multiple time regions. The associating allows electrodermal activity to be related to self-reported pain (SRP) data to derive an objective pain measurement.

### RELATED APPLICATIONS

This application claims the benefit of U.S. provisional patent application “Pain Analysis Using Electrodermal Activity” Ser. No. 61/845,932, filed Jul. 12, 2013. The foregoing application is hereby incorporated by reference in its entirety.

### FIELD OF ART

This application relates generally to pain analysis, and more particularly to pain analysis using electrodermal activity.

### BACKGROUND

When a person in need of medical treatment first comes into contact with a health professional such as a doctor, a physiotherapist, or a nurse, the person generally tries to verbally describe his or her pain in order to allow the medical professional to preliminary diagnose the patient's condition and suggest a suitable treatment. However, certain situation can complicate such an interaction. For example, certain people are more resistant to pain than others, while other people who deal with chronic pain can become desensitized to the pain after extended periods of time and describe the pain in overly mild terms. Such varying, highly subjective descriptions of pain presented to a diagnostician can complicate a quick and exact identification of a person's ailment or injury. While diagnostic procedures (e.g., MRI, x-ray, ultrasound, etc.) provide data allowing for accurate determinations of physiological conditions such as damage to bones or tissue, pain is almost always measured by asking for patient feedback.

In situations where analgesics are being dispensed, particularly in Patient Controlled Analgesia (PCA), there is a pervasive opportunity for unwarranted and excessive medication administration. Thus, the process of PCA, which requires both the attention of trained nursing staff and the orders of a physician, might not achieve properly adjusted parameters in time to minimize patient pain. Furthermore, relying heavily on patient reports of pain provides opportunities for patients to manipulate physicians in an effort to obtain more pain medication than necessary. Additionally, under-treatment of acute, and especially chronic pain can occur, especially in cases where a routine physical examination cannot identify the source of the pain. Even in emergency departments where staff frequently evaluate and/or treat patients with traumatic injuries such as broken legs or dislocated shoulders, the staff may still undertreat or over-treat with analgesia, often selecting the level of medication based on little more than past experience and the assumption that different patients will respond similarly to similar dosages.

Any attempts at objectively identifying and treating pain have been hampered by the lack of means to reliably measure pain. Usually when a patient complains of pain, physicians attempt to determine the nature of pain by asking the patient to first describe the pain. Similarly, any attempts made to determine the severity of the patient's pain also rely on the patient to identify the severity of his or her own pain. Then, the most appropriate treatment for the patient's pain is determined, including stipulating appropriate types of pain-relief medications. Such treatment is at best experimental and at worst incorrect, leading to various unwanted side effects. In some patients who complain of unusual pain or who feign pain, the whole treatment might fail, or at least delay relief by many days or weeks. In the case of chronically painful conditions such as arthritis, back pain, headache or migraine headache pain, and abdominal pain from various causes, determining appropriate doses of medications and periodically identifying beneficial changes in medications becomes crucial. As different people often use different values to identify perceived pain levels and different adjectives to describe the pain, relying on patient feedback for pain determination is highly subjective.

### SUMMARY

Disclosed are systems and methods to provide an objective assessment of pain by utilizing a combination of electrodermal activity (EDA) and self-report data. Patients are monitored with a variety of biosensors, which include EDA sensors and can further include other sensors, such as sensors capable of capturing heart rate, skin temperature, and accelerometer data, among others. EDA data is monitored over multiple time regions, and a relationship between self-reported pain (SRP) and the EDA data is developed. A computer-implemented method for analyzing physiology is disclosed comprising: obtaining electrodermal activity for an individual and evaluating a pain level based on the electrodermal activity for the individual wherein the evaluating covers a plurality of regions of time and includes relating the electrodermal activity to self-report data for the individual.

In embodiments, a computer program product embodied in a non-transitory computer readable medium for analyzing physiology can comprise: code for obtaining electrodermal activity for an individual and code for evaluating a pain level based on the electrodermal activity for the individual wherein the evaluating covers a plurality of regions of time and relates the electrodermal activity to self-report data for the individual. In some embodiments, a computer system for physiology analysis can comprise: a memory which stores instructions and one or more processors coupled to the memory wherein the one or more processors, when executing the instructions which are stored, are configured to: obtain electrodermal activity for an individual and evaluate a pain level based on the electrodermal activity for the individual wherein evaluation covers a plurality of regions of time and relates the electrodermal activity to self-report data for the individual. In embodiments, a computer-implemented method for physiology analysis can comprise: receiving electrodermal activity which was obtained from an individual and evaluating a pain level based on the electrodermal activity for the individual wherein the evaluating covers a plurality of regions of time and relates the electrodermal activity to self-report data for the individual. In some embodiments, a computer-implemented method for physiology analysis can comprise: capturing electrodermal activity for an individual and sending the electrodermal activity to a server device for the evaluating of a pain level based on the electrodermal activity for the individual wherein the evaluating covers a plurality of regions of time and relates the electrodermal activity to self-report data for the individual. In embodiments, a computer-implemented method for physiology analysis can comprise: receiving an evaluation of a pain level based on electrodermal activity for an individual wherein the evaluation covers a plurality of regions of time and relates the electrodermal activity to self-report data for the individual, and displaying a result of the evaluation of the pain level.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for pain analysis.

FIG. 2 is a flow diagram for detailed evaluation.

FIG. 3 is a flow diagram for relating and norm usage.

FIG. 4 shows an example response during stimulus.

FIG. 5 shows example average skin conductance level response.

FIG. 6 shows an example biosensor on a person.

FIG. 7 is a system diagram for pain analysis.

### DETAILED DESCRIPTION

Disclosed embodiments provide an objective measure of perceived pain. Electrodermal activity reflects autonomic nervous system activity, and thus provides insightful ways to assess an individual's physical or mental state. In particular, some electrodermal activity can exhibit signatures or characteristics associated with a certain physiological condition. Many such conditions are described herein, and still others will be appreciated, including pain, anxiety, panic attacks, epileptic seizures, sleep disorders, heart attacks, or the like. In disclosed embodiments, the usefulness of EDA data is extended by relating the collected data to self-reported pain (SRP) data to derive an objective pain measurement. The usefulness of such an objective pain measurement can be appreciated across numerous medical and pharmaceutical applications. For medical applications, embodiments of objective pain measurement could aid a physician in dispensing the appropriate level of pain medications, so that a patient is neither over nor under-medicated. As certain types of pain medication carry a high potential for addiction, limiting excessive dosage of such medications could prove extremely beneficial to a patient's wellbeing. Further, pharmaceutical applications of objective pain measurement as disclosed herein could aid researchers in determining the pain caused by administration of various medicines and assist in gauging the effectiveness of analgesics and local anesthetics.

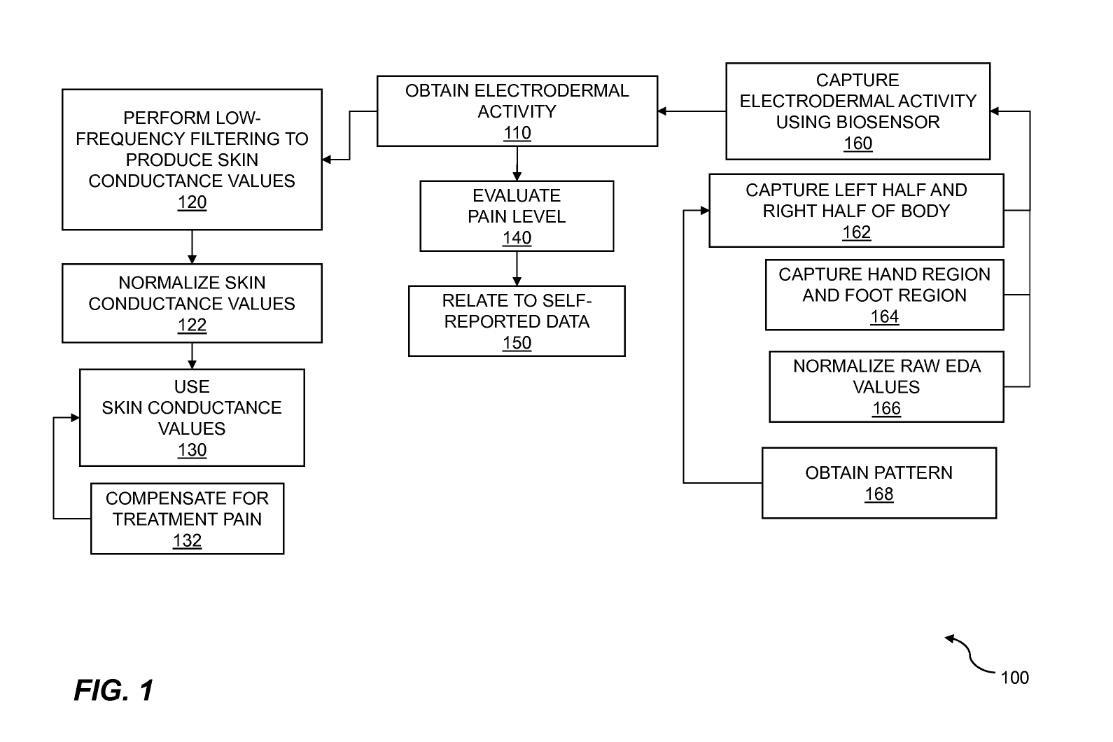

FIG. 1 is a flow diagram for pain analysis. The flow 100 describes a computer-implemented method for analyzing physiology. The flow 100 comprises obtaining electrodermal activity 110 for an individual. The flow 100 can further comprise performing low-frequency filtering to produce skin conductance values 120. The skin conductance values can be measured in micro-Siemens or in any other appropriate value. The flow 100 can include normalization of the skin conductance values 122 to ensure the values are within predetermined ranges for further use and evaluation. Next, the flow 100 comprises using the skin conductance values 130 for pain assessment by implementing any relevant equations for determining pain levels from conductivity measurements. The flow 100 can further comprise compensating for treatment pain 132 and compensating for anxiety, as such compensations can increase the accuracy of pain determinations. For example, taking a measure of a patient's skin conductance values prior to administration of treatment allows compensation to be performed for the patient's pre-treatment anxiety level. That is, when a patient is expecting a painful needle, his or her electrodermal activity may increase in anticipation of pain. Further, in some applications it may be useful to compensate for treatment pain 132. Certain treatments carry a component of pain independent of pain caused, or alleviated, by administered medications. For example, a first component of pain could be caused by the needle entering the patient's body, and a second component of pain could be caused by administration of the medication (e.g. by pushing on a syringe). Compensating for the first component of pain (the needle entering the body) can allow for determining a more objective measurement of the pain caused by the medication. In embodiments, compensating for anxiety and needle pain can allow an accurate identification of the pain caused by the administration of the medication. Compensation can be performed for various other factors as well, such as body temperature, body type (e.g. endomorph, mesomorph, ectomorph), and ambient room temperature and humidity.

The obtaining of the electrodermal activity can be accomplished by capturing the electrodermal activity using at least one biosensor 160. In some embodiments, a biosensor is placed at one or more regions of a patient's body, including the left hand region, the right hand region, the left foot region, and the right foot region. The hand region can comprise the hand and the wrist and the foot region can comprise the foot and the ankle In some embodiments, a biosensor is placed on the left and/or right palm of a patient. A biosensor can also be placed on or near the sternum of a patient. The biosensor can include one or more metal electrodes fastened to a patient. The capturing can include capturing electrodermal activity from a left half of a body and a right half of a body 162. The capturing can include capturing electrodermal activity from a hand region of a body and a foot region of a body 164. Differences in electrodermal activity between the left half of a body and the right half of a body can provide an indication of the general location of the pain. The general location of the pain can also be related to a location of stimuli. For example, an injection administered in the left arm may induce more electrodermal activity on the left side than on the right side.

The flow 100 includes evaluating a pain level 140 based on the electrodermal activity for the individual wherein the evaluating covers a plurality of regions of time and includes relating the electrodermal activity to self-report data for the individual. In embodiments, the plurality of regions of time comprises three time regions: a time before stimulus, a time from stimulus to a self-reported maximum pain level, and a time from the self-reported maximum to a certain subsequent time. A first region can be called a warning region, and is defined from a predetermined period prior to stimulus until the stimulus's starting point. For example, in the case of an injection, the warning region can comprise a time from 120 seconds prior to the administration of the injection until the point at which the injection is administrated. A second region can comprise the time from the stimulus starting point until a maximum self-report pain (MSRP) level. The MSRP level is based on various methods of soliciting user feedback, such a pain reporting scale of 1 to 10, where the individual is asked to rate from a value of 1 (no pain) to 10 (the highest possible pain level under the circumstances). The regions of time can also be based on one or more of physiological data and psychometric data where the psychometric data can also include self-reported pain (SRP) data. Relating a pain level to self-reported data 150 can be helpful in nuancing electrodermal readings.

The flow 100 can further comprise performing normalization of raw values from the electrodermal activity 166 to compensate for a baseline electrodermal activity before treatment. The normalization can include subtracting mean electrodermal activity from the raw values of the electrodermal activity and dividing the resulting electrodermal activity value by a standard deviation. In some embodiments, the normalized data has a zero mean and a standard deviation of one.

The flow 100 can comprise obtaining a fundamental pattern 168 present in the electrodermal activity from the left and right body regions for each individual from a plurality of people and performing signature analysis on the fundamental patterns. The fundamental pattern for each individual can include one or more temporally spaced peaks in electrodermal activity, and also can include measurement of the number of peaks per minute, rise time and fall time for the peaks, elevated time duration, bilateral difference, difference between dominant and non-dominant sides, peak to valley range, storming activity, areas under one or more time regions of the curve, or the like. In embodiments, accelerometer data can be collected on the plurality of people and a characteristic of the accelerometer data can be identified and used in conjunction with the electrodermal activity pattern. The characteristic pattern of the electrodermal activity and the characteristic of the accelerometer data can be used separately or together for inferring pain levels. Embodiments can include a computer-implemented method comprising: obtaining electrodermal activity for a plurality of people, performing signature analysis on the electrodermal activity to identify a characteristic of the electrodermal activity that corresponds to a pain, and evaluating a pain level based on the electrodermal activity for the plurality of people. Various steps in the flow 100 may be changed in order, repeated, omitted, or the like without departing from the disclosed concepts. Various embodiments of the flow 100 may be included in a computer program product embodied in a non-transitory computer readable medium that includes code executable by one or more processors.

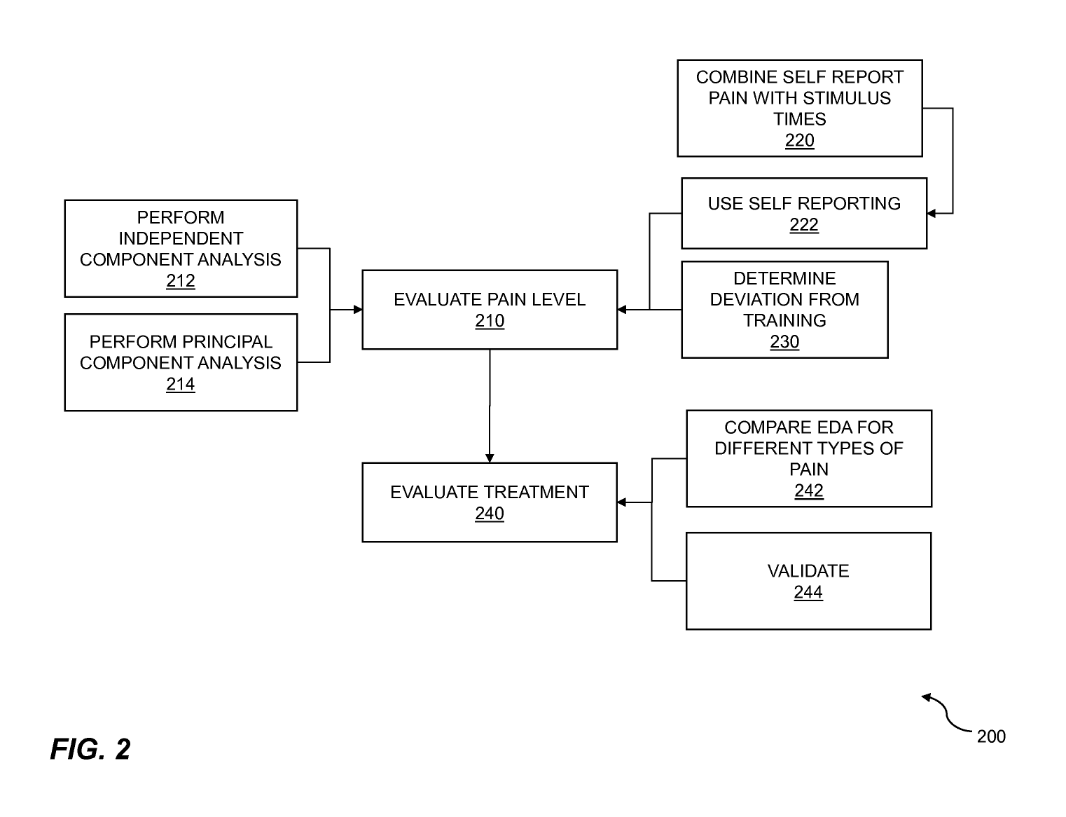

FIG. 2 is a flow diagram for detailed evaluation. The flow 200 comprises evaluating a pain level 210. The evaluating of a pain level can include evaluating electrodermal activity and/or skin conductance values. The flow 200 can include combining self-reported pain response with a pain stimulus start time and end time 220 to place self-reported pain responses within defined regions of objectively identified pain. In this way, stimuli that produce a delayed pain sensation can be identified. For example, the pain from a stimulus may not reach its maximum level until several seconds or more after the stimulus is applied. The flow 200 can also comprise using self-reporting 222 as part of the evaluating pain response. As noted earlier, some embodiments use three regions of time to define a pain event, and the self-reporting can be used across these three time regions. The self-reporting can comprise a user selecting a numerical value based on a numerical rating scale. In some embodiments, an eleven-value scale is used, where a value of 0 indicates no pain, a value from 1 to 3 indicates mild pain (nagging or annoying pain), a value from 4 to 6 indicates moderate pain (interfering with daily tasks), and a value from 7 to 10 indicates severe pain (disabling, unable to perform daily tasks). Other scales can be used such as a 1-100 scale. Such a scale can be continuous or can use integer values. In some embodiments, an electrical visual analog scale (EVAS) is used. The use of self-reporting to denote a time of maximum pain can allow electrodermal activity or skin conductance values to be evaluated at or near the time of maximum pain, providing a way to correlate and quantify objective pain measurements in view of subjective pain. In some cases, peak electrodermal activity or skin conductance values may lag or lead the time of the maximum self-reported pain.

The flow 200 may comprise determining deviations in electrodermal activity from a training session 230 where no treatment is administered. For example, it can be desirable to compensate an individual's skin conductance level values for anxiety experienced by the individual. Towards this end, electrodermal activity can be collected for one or more patients where no treatment is administered, or a placebo treatment designed to match the psychological effects of the actual treatment is given. In this way, compensation for electrodermal activity due to factors other than treatments, such as anxiety, can be included as part of the pain analysis, so that pain due to the treatment (treatment pain) can be accurately assessed. The treatment pain can include pain directly stemming from needle usage, pain caused by the needle injecting a pain-causing substance, or the like. Understanding pain stimuli such as needle usage can allow researchers and medical professionals to account for needle pain and avoid biasing other analyses.

The flow 200 can further comprise performing a low-frequency filter on the electrodermal activity to produce skin conductance level values, and using the skin conductance level values in the evaluating. The electrodermal activity values can be divided into Tonic (low frequency) or skin conductance level (SCL), and Phasic (high frequency) or skin conductance response (SCR) components. SCL measurements can describe electrodermal activity through rather long intervals, often over a period of time such as tens of seconds to tens of minutes. In the relatively longer measurement intervals of SCL, electrodermal activity tends to be spiky, with substantial high frequencies. Hence, signal conditioning such as low-pass filtering can be helpful to isolate true skin conductance values (levels) from noise, and can be useful for obtaining accurate data in order to compare multiple trials from multiple patients, for example. In some embodiments, the low frequency filter can include a fifth order, zero-phase, low-pass Butterworth filter. The Butterworth filter is a type of signal processing filter designed to produce as flat a frequency response as possible in the pass band. In some embodiments, the low frequency filter includes a cutoff frequency of 0.05 Hz. The filtering can also include estimating a skin conductance level for each one-minute epoch using a moving average filter. Additionally, the filtering can include evaluating the skin conductance level values for a pain stimulus experienced by the individual. For example, a sharp elevation in skin conductance can be related to a pain level, based on data collected from previous patients. The relating can include comparing electrodermal activity during these regions for different types of pain stimuli to evaluate the effects of a treatment.

The flow 200 can further comprise evaluating a treatment 240 based on the electrodermal activity. The evaluating a treatment 240 can include comparing skin conductance levels between multiple treatment scenarios and determining if one or more of those scenarios results in reduced skin conductance levels. Reduced maximum skin conductance levels can be normalized using a filter or another appropriate method to ensure data accuracy. If the normalized skin conductance levels still show a reduction in conductivity for a certain treatment compared to other treatments, the treatment associated with reduced conductivity can be correlated with less intense patient reactions to the treatment. Medical professionals, among others, can find objective data on patient reactions helpful in adjusting a treatment protocol, improving or changing dosages of a medication, or eliminating certain medication delivery methods, among numerous possible applications.

Continuing, the flow 200 can further comprise comparing electrodermal activity for different types of pain 242. As previously discussed, reduced skin conductance can correlate with a reduced pain level for a certain treatment type, though pain measurement is not limited to treatment pain, rather pain can be measured for both for ailments and treatments. Ailment pain types can include, but are not limited to, acute pain, chronic pain, muscle pain, joint pain, dental pain, sinus pain, and stomach pain. Treatment pain types can include needle pain. As before, the practical applications for methods to actively measure and differentiate between treatment and ailment pain are varied and numerous, across a variety of fields.

The flow 200 can further comprise validating 244 the relating to distinguish the drug treatment from a placebo, including performing a statistical significance test. Again, distinguishing between different sources of patient pain (in this case pain from a drug administration and pain from either the delivery method or simply from anticipation of a treatment) can prove extremely useful, and can be measured using mathematical tests on the gathered skin conductance data. The statistical significance test may include a Wilcoxon Rank-Sum test. A Wilcoxon test is a non-parametric statistical hypothesis test that can be used in comparing related samples, matched samples, or repeated measurements on a single sample. This test helps evaluate if samples have differing population mean ranks The evaluating may include performing principal component analysis 214. Principal component analysis is a mathematical procedure where orthogonal transformation is used in converting a possibly correlated variable observation set into a set of values with linearly uncorrelated variables. These are, in turn, called principal components. The evaluating can include performing independent component analysis 212. Independent component analysis includes statistical and computational techniques that reveal hidden factors that underlie sets of random variables, measurements, or signals. Various steps in the flow 200 may be changed in order, repeated, omitted, or the like without departing from the disclosed concepts. Various embodiments of the flow 200 may be included in a computer program product embodied in a non-transitory computer readable medium that includes code executable by one or more processors.

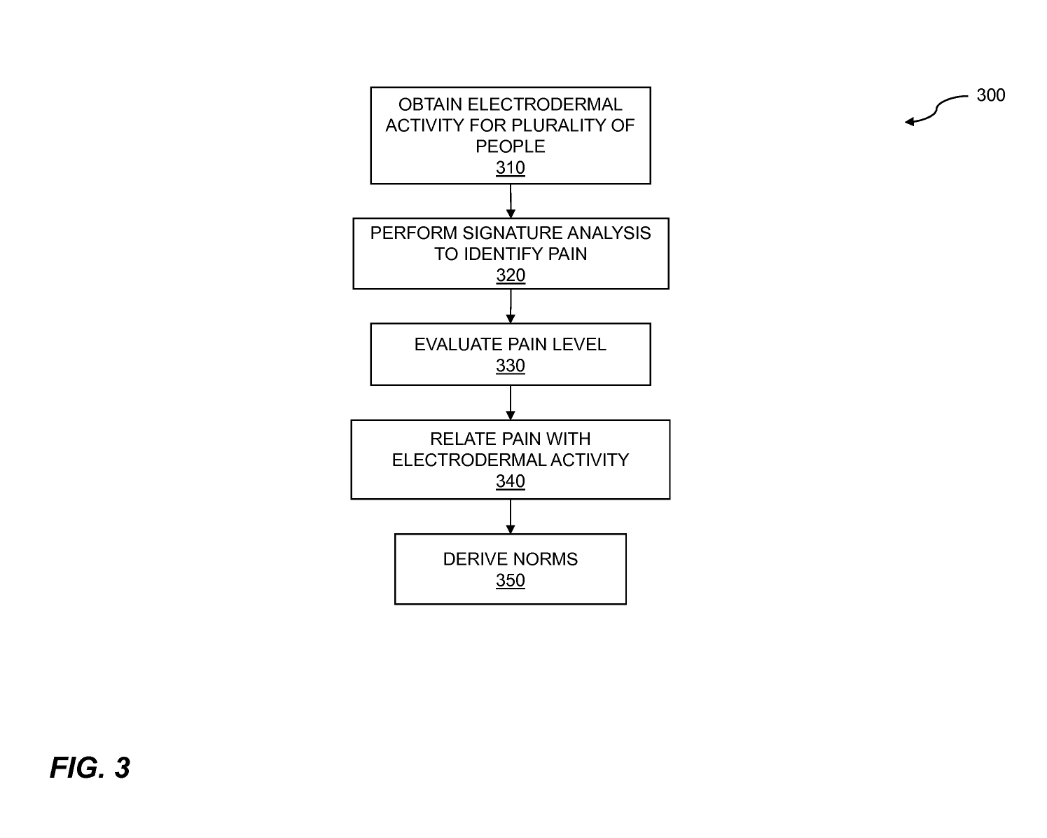

FIG. 3 shows a flow diagram for relating pain with electrodermal activity and norm derivation. The flow 300 comprises obtaining electrodermal activity for a plurality of people 310. For example, a group of 30 to 40 people can be subjected to a similar treatment stimulus and electrodermal activity collected for each of the plurality. The electrodermal activity for each of the people can be processed—with, for example, low-pass filtering—normalized, and averaged, to provide an overall electrodermal signature for the treatment. By gathering data from a group of people in known contexts or experiencing known physiological conditions, it becomes possible to extract signatures in electrodermal activity data by noting relationships, similarities, and differences in data sets gathered from different groups undergoing the same stimulus. That is, signature reactions in the electrodermal activity data of an individual can be determined by comparing the individual's data signature with the signatures of a plurality of other people undergoing the same stimulus. When signatures are found in the electrodermal activity data of the individual, the physiological condition of the individual at the time the signature appeared in the electrodermal activity data can be determined or inferred. Specifically, in the case of flow 300, the physiological condition of the individual at the time a signature of note is observed can be matched with a known pain profile associated with the same signature in the collected data from the group of people. The pain profile can include a level of pain, as well as a location of the pain, the sharpness of the pain, and the frequency of the pain.

To further aid in the identification, the flow 300 can comprise performing signature analysis 320 on the electrodermal activity to identify a characteristic of the electrodermal activity that corresponds to a pain. The characteristic can include a spike in skin conductance values at or near a maximum self-reported pain time. The flow 300 can also comprise evaluating a pain level 330. The amplitude of the spike in skin conductance values can be related to a pain level, where a higher spike amplitude relates to a higher level of pain. The flow 300 can further comprise relating pain experienced by the plurality of people with the electrodermal activity 340 for the plurality of people. This can include identifying relative maxima of skin conductance values proximal to a maximum self-reported pain level. The flow 300 can include deriving norms 350 based on the electrodermal activity from the plurality of people and using the norms in evaluating a treatment. Some analysis can be performed using a web service. Various steps in the flow 300 may be changed in order, repeated, omitted, or the like without departing from the disclosed concepts. Various embodiments of the flow 300 may be included in a computer program product embodied in a non-transitory computer readable medium that includes code executable by one or more processors.

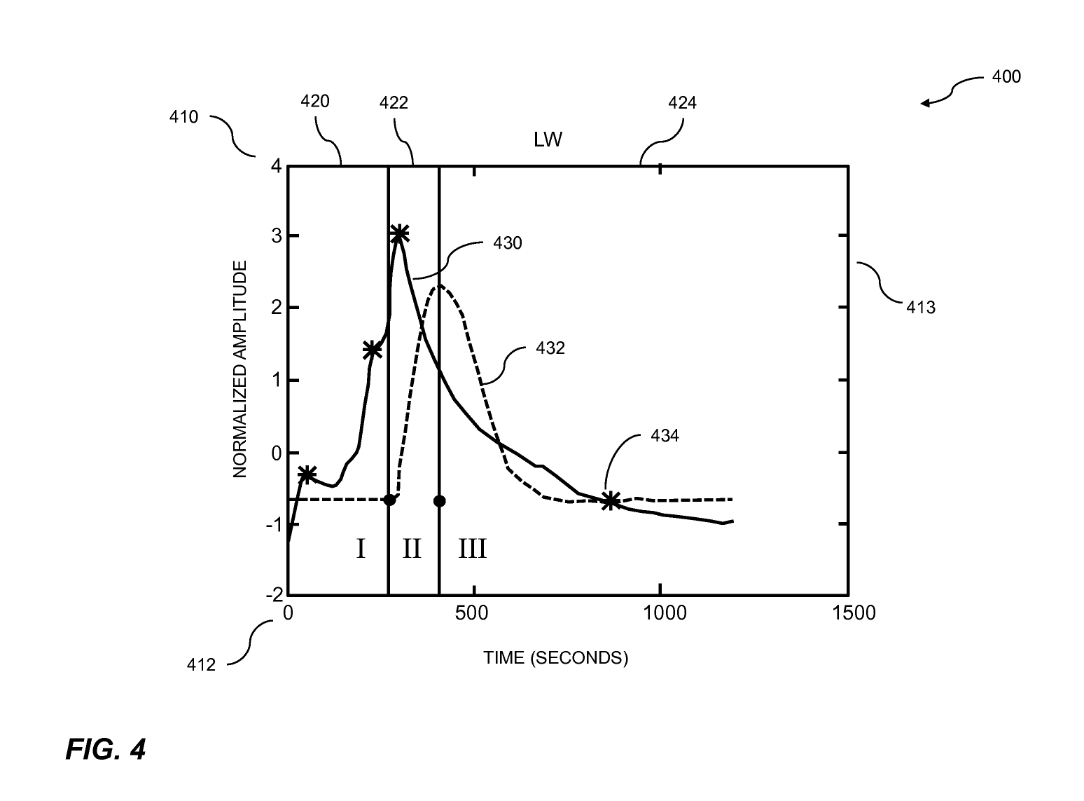

FIG. 4 shows a graph 400 of an example response during stimulus. The graph 400 comprises a horizontal (X) axis 412, which shows time. In embodiments, the time can be displayed in seconds, minutes, or another unit of time. The graph 400 also comprises a first vertical (Y) axis 410. In the example shown, the first Y-axis displays a normalized amplitude of skin conductance. The graph 400 further comprises a second vertical (Y) axis 413, which displays a self-reported pain index. In the example shown, the graph 400 relates self-reported pain to skin conductance levels. In embodiments, the relating includes combining self-reported pain response with a pain stimulus start time and end time to define regions of pain responses. The relating can also include comparing electrodermal activity during the response regions for different types of pain stimuli to evaluate the effects of a treatment.

In the example 400, curve 430 represents skin conductance values as a function of time and curve 432 represents self-reported pain values as a function of time. The graph 400 can be divided into multiple regions of time. In the example shown, and as was discussed earlier, three regions of time are used to delineate skin conductance responses in relation to certain periods surrounding the application of a stimulus. A first region 420, from the three regions of time, can include measurements of electrodermal activity during a warning period from a predetermined beginning of an event to a time before a stimulus. The warning period can extend from a predetermined beginning of an event at time t0 to time t1. The stimulus can include the administration of a drug via a syringe. In some embodiments, the duration of time for region 420 can range from about 100 seconds to about 500 seconds. A second region 422 can include the electrodermal activity during a stimulus period from a time of a stimulus beginning to a time t2 of maximum self-reported pain. A third region 424 can include the electrodermal activity during a pain recovery period from a time after self-reported maximum pain to the end of an event. In some embodiments, the end of an event is signified by the expiration of a fixed duration of time. The duration of time for the third region 424 can range from about five hundred seconds to about a thousand seconds. In some embodiments, the end of an event can also be signified by reaching a steady state of self-reported pain, as indicated at point 434 on curve 432. The maximum amplitude M of curve 430 within second time region 422 can be used to ascertain a pain level experienced by a patient. A larger amplitude M can relate to a higher level of perceived pain.

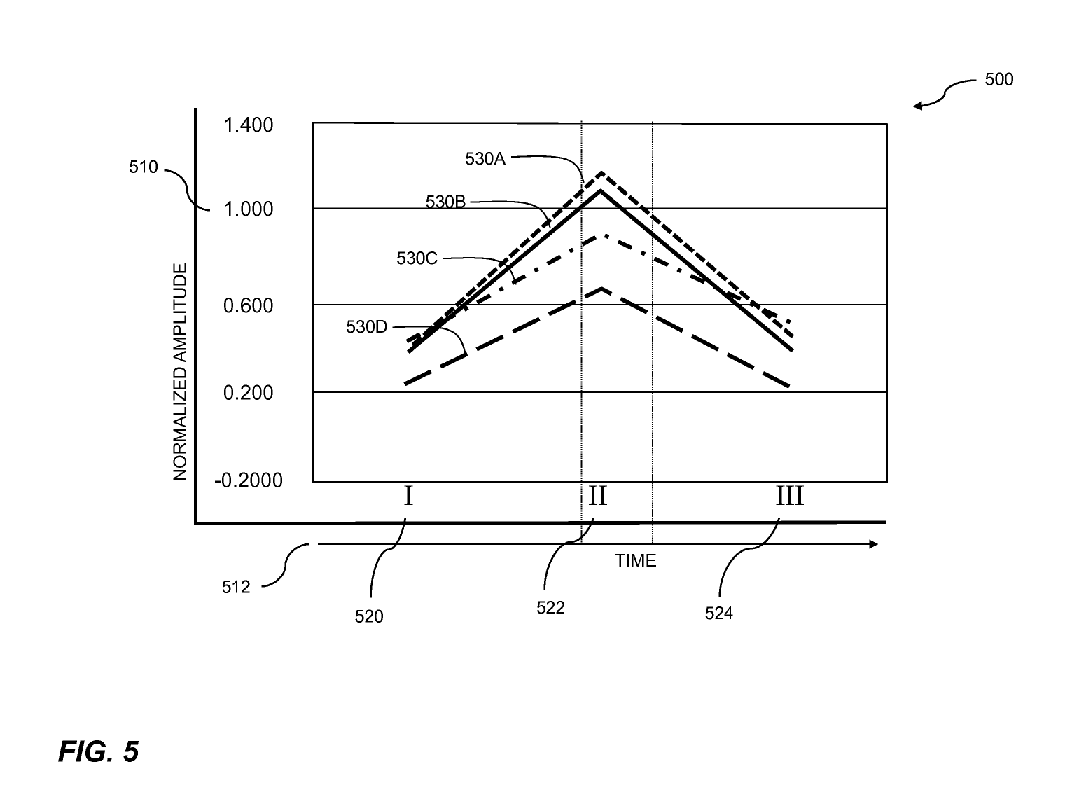

FIG. 5 shows a graph 500 of an example average skin conductance level response. A horizontal (X) axis 512 indicates time. As was the case in FIG. 4, three time regions are indicated. In the graph 500 shown, region 520 is a warning region spanning from a predetermined time to the time when a stimulus is introduced. Region 522 is a second region containing, in embodiments, a record of the electrodermal activity during a stimulus period spanning from the beginning of the stimulus to a time of maximum self-reported pain. Region 524 can include the electrodermal activity during a pain recovery period from a time beginning after the self-reported maximum and continuing to a specified end of an event. A vertical Y-axis 510 represents a normalized amplitude for skin conductance levels. A first curve 530A, a second curve 530B, a third curve 530C, and a fourth curve 530D represent skin conductance values as a function of time. Each curve represents skin conductance values from a different patient. Data from a plurality of people can be aggregated to form a generic skin conductance response curve for a particular treatment. The aggregation can include averaging data. In some embodiments, some data is discarded, and not included in the averaging. For example, amongst the various samples collected, the minimum and maximum values of the peak skin conductance value within the second region 522 may be discarded as outliers.

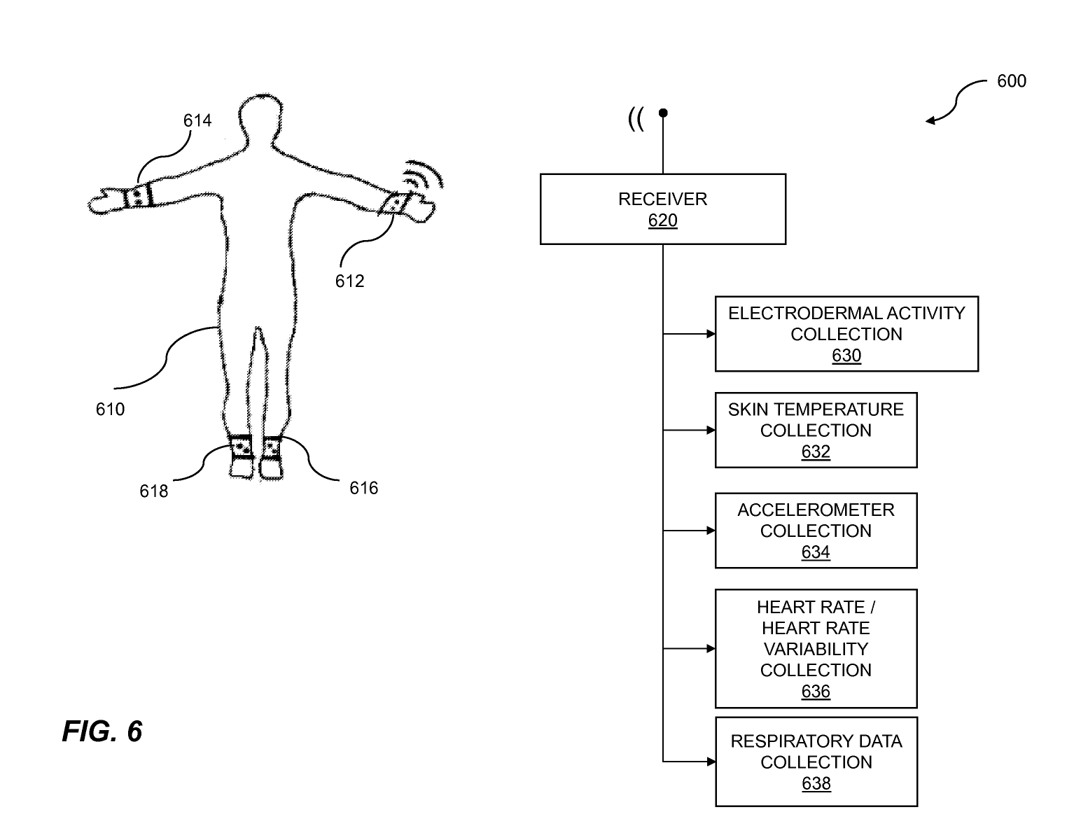

FIG. 6 shows a diagram 600 of example biosensors on a person 610. One or more biosensors can be attached to the person in various ways. A biosensor 612 can be used on a right hand region of the person 610. A biosensor 614 can be used on a left hand region of the person 610. A biosensor 616 can be used on a right foot region of the person 610. A biosensor 618 can be used on a left foot region of the person 610. The hand regions can include the hand and the wrist. The foot regions can include the foot and the ankle In embodiments, each biosensor transmits information to a receiver 620. The transmission of information can be wireless. In some embodiments, the transmission protocol can include infrared, Bluetooth®, ZigBee®, or any other appropriate form of wired or wireless communication. Electrodermal activity collection 630 can be performed based on the information received by receiver 620. Embodiments can perform skin temperature collection 632. The skin temperature collection can be performed with a biosensor, and/or via imaging techniques. Embodiments can include accelerometers worn by the person 610, and can include accelerometer data collection 634. The accelerometer data collection 634 can further quantify pain that the individual is experiencing. For example, the accelerometer data collection 634 can indicate hand or wrist movements consistent with intense pain, or a lack of hand or wrist movement consistent with a lack of intense pain. The collection can include heart rate and/or heart rate variability data collection 636. This data can be collected via biosensors and/or via non-contact methods, such as video imaging techniques. Embodiments can include respiratory data collection 638, such as breathing rate. The skin temperature data, accelerometer data, heart rate data, heart rate variability data, and/or respiratory data can be used as supplemental data to derive a pain value. The pain value can thus be a function of more than collected electrodermal activity and can be expressed as a function of one or more sources of supplemental data.

Embodiments of each of these structures can be implemented in hardware, software, a combination of hardware and software, and the like. Embodiments of each of these structures can receive data corresponding to two through four of the sensors, such as the right wrist sensor 614, the left wrist sensor 612, the right ankle sensor 618 and the left ankle sensor 616, and can track differences between any two of them. In embodiments, the sensors can be attached to the individual in pairs, with one sensor of a pair on a left appendage and the other sensor of the pair on a right appendage. The left and right appendages could be palms, hands, wrists, forearms, elbows, arms, feet, ankles, legs, knees, thighs, or the like. Sensors could also be placed on the sternum, head, or elsewhere.

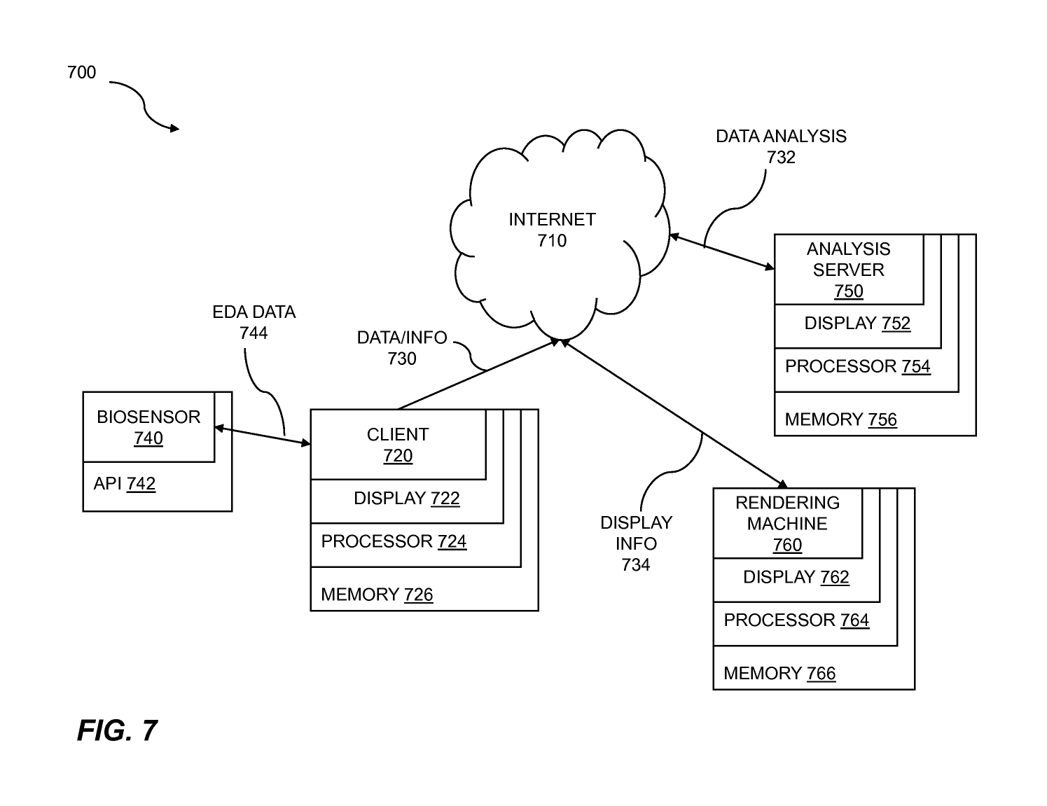

FIG. 7 is a system diagram showing a system 700 for pain analysis. The Internet 710, intranet, or other computer network can be used for communication between the various devices. The system 700 includes a biosensor 740 which can also include an electrodermal sensor. The biosensor 740 can further include one or more of a heart rate sensor, a respiratory rate sensor, a skin temperature sensor, and an accelerometer. The biosensor 740 can include an application programming interface (API) 742. The API 742 can provide a protocol through which software components can interface with the biosensor 740. The software components can be provided by third parties and the software components can control and use certain aspects of the biosensor 740. A library of software components or plug-in routines can be used to aid in electrodermal activity analysis and physiology analysis, and to provide mental state analysis enablement with the biosensor 740. In embodiments, the biosensor 740 transmits electrodermal data 744 to a client machine 720.

The client machine 720 can include a memory 726 which stores instructions, and one or more processors 724 attached to the memory 726 as well as a display 722. The display 722 can be any electronic display, including but not limited to, a computer display, a laptop screen, a net-book screen, a tablet screen, a cell phone display, a mobile device display, a remote with a display, a television, a projector, or the like. The client machine 720 can include one or more user input devices such as a keyboard, mouse, joystick, touchpad, wand, motion sensor, and other input means. The client machine 720 can also receive psychometric data, such as self-reported pain data via a user input device. The client machine 720 can communicate with the analysis server 750 over the Internet 710, some other computer network, or by other method suitable for communication between two computers.

The analysis server 750 can have an Internet connection for receiving data and transmitting data analysis 732, a memory 756 which stores instructions, and one or more processors 754 attached to the memory 756. The analysis server 750 can further include a display 752. The analysis server 750 can receive data 730 from multiple clients 720 and aggregate data in a process that can include filtering, averaging, and other mathematical manipulations. The analysis server 750 can derive skin conductance levels from the electrodermal activity. The server 750 can perform relating of electrodermal activity to pain. The relating can also be performed by a web service. The server 750 can implement multiple web services for various data analysis functions including, but not limited to, filtering, averaging, peak identification, and derivation of a numerical pain score. The web services can support an interface and can include a server that is remote to the individual and/or cloud-based storage. Web services can include a web site, an ftp site, or a server which provides access to a larger group of analytical tools for analyzing pain profiles. Other data analysis functions are possible as well. The analysis server 750 can transmit the data analysis 732 via the Internet 710. The web services can also be a conduit for collected data as it is routed to other parts of the system. The data can include a serialized object in a form of JavaScript Object Notation (JSON). The method of system 700 can further comprise deserializing the serialized object into a form for a JavaScript object. The web services can be implemented by a server or a distributed network of computers. The web services can provide a means for a user to log in and request information and analysis. The information request can take the form of analyzing a pain level for an individual in light of various other sources of information, or can be based on a group of people who relate to the pain level for the individual of interest. In some embodiments, the web services can provide for the forwarding of data which was collected to one or more processors for further analysis.

Data can be retrieved through accessing the web services and requesting data which was collected for an individual. Data can also be retrieved for a collection of individuals, for a given time period, or for a given treatment. Data can be queried to find matches for a specific treatment, for a given pain profile or pain level, or for an individual or group of individuals. Associations can be found through queries and various retrievals which can prove useful in a hospital or pharmaceutical environment. Queries can be made based on key word searches, based on time frame, or based on experience.

The analysis which is received from the analysis server 750 can be based on specific access rights. For example, a machine receiving the analysis can be authenticated and granted access to the analysis based upon business rules. As another example, a user name and password can be provided to the analysis server 750 and the analysis server 750 can validate the user name and password prior to the analysis server 750 transmitting the analysis. By way of example, and not of limitation, a clinical director might be able to view aggregated responses and/or clusters of information while an individual might only be able to see their own personal data. A variety of examples of access rights and ways of enforcing access rights will be appreciated.

The system 700 can include a rendering machine 760. The rendering machine can include one or more processors 764 coupled to a memory 766 to store instructions and a display 762. The rendering machine 760 can receive display information 734 received over the Internet 710 or another network. The display information 734 can include data analysis from analysis server 750 and/or data from one or more clients 720, and can render an output to the display 722. The rendered output can include, but is not limited to, a numerical pain value. In some embodiments, the numerical pain value can range from 0 to 100, with 0 representing no pain and 100 representing maximum pain. The rendered output can include an adjective describing the pain level such as “mild,” “moderate,” “intense,” and “severe.” The rendered output can include, but is not limited to, a graphical representation of the pain as a function of time. The graphical representation can be based on electrodermal activity data 744 collected from biosensor 740. The rendered output can also include a location of pain within the body, such as left side, right side, left leg, right leg, left arm, right arm, entire body, lower extremities, and upper extremities. The rendered output can further comprise recommending a course of action based on the numerical pain value. For example, the rendered output might recommend increasing or decreasing a dosage of pain medication based on the numerical pain value. The rendered output can further comprise recommending cessation of a pain medication when the numerical pain value is below a predetermined threshold value. The system 700 can enable a computer-implemented method for physiology analysis comprising receiving electrodermal activity which was obtained from an individual, evaluating a pain level based on the electrodermal activity for the individual wherein the evaluating covers a plurality of regions of time and relates the electrodermal activity to self-report data for the individual.

The system 700 can enable a computer-implemented method for physiology analysis which further comprises: capturing electrodermal activity for an individual, sending the electrodermal activity to a server device for evaluating of a pain level based on the electrodermal activity for the individual wherein the evaluating covers a plurality of regions of time and relates the electrodermal activity to self-report data for the individual. The system 700 can enable a computer-implemented method for physiology analysis that further comprises receiving an evaluation of a pain level based on electrodermal activity for an individual wherein the evaluation covers a plurality of regions of time and relates the electrodermal activity to self-report data for the individual, and displaying a result of the evaluation of the pain level. The system 700 may include a computer program product embodied in a non-transitory computer readable medium for analyzing physiology comprising: code for obtaining electrodermal activity for an individual and code for evaluating a pain level based on the electrodermal activity for the individual wherein the evaluating covers a plurality of regions of time and relates the electrodermal activity to self-report data for the individual.

Each of the above methods may be executed on one or more processors on one or more computer systems. Embodiments may include various forms of distributed computing, client/server computing, and cloud based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the forgoing examples should not limit the spirit and scope of the present invention; rather it should be understood in the broadest sense allowable by law.

What is claimed is:

1. A computer-implemented method for analyzing physiology comprising:
obtaining electrodermal activity for an individual; and
evaluating a pain level based on the electrodermal activity for the individual wherein the evaluating covers a plurality of regions of time and includes relating the electrodermal activity to self-report data for the individual.

2. The method of claim 1 wherein the plurality of regions of time include a time before stimulus, a time from stimulus to a self-reported maximum pain level, and a time from the self-reported maximum pain level to a subsequent time.

3. The method of claim 1 further comprising performing normalization of raw values from the electrodermal activity to compensate for a baseline electrodermal activity before treatment.

4. The method of claim 3 wherein the normalization includes subtracting mean electrodermal activity from the raw values of the electrodermal activity and dividing a resulting electrodermal activity value by a standard deviation.

5. The method of claim 1 further comprising performing a low frequency filter on the electrodermal activity to produce skin conductance level values and using the skin conductance level values in the evaluating.

6. The method of claim 5 further comprising evaluating the skin conductance level values for pain stimulus experienced by the individual.

7. (canceled)

8. The method of claim 5 wherein the low frequency filter includes a fifth order, zero-phase, low-pass Butterworth filter.

9. The method of claim 5 wherein the low frequency filter includes a cutoff frequency of 0.05 Hz.

10. The method of claim 1 further comprising using self-reporting as part of the evaluating a pain level with respect to three regions of time wherein the three regions of time comprise the plurality of regions of time.

11. The method of claim 10 wherein a first region, from the three regions of time, includes the electrodermal activity during a warning period from a beginning of an event to a time before a stimulus.

12. The method of claim 10 wherein a second region includes the electrodermal activity during a stimulus period from a time of a stimulus beginning to a time of self-reported maximum pain.

13. The method of claim 10 wherein a third region includes the electrodermal activity during a pain recovery period from a time after self-reported maximum pain to an end of an event.

14. The method of claim 1 wherein the obtaining of the electrodermal activity is accomplished by capturing the electrodermal activity with use of at least one biosensor wherein the capturing includes capturing electrodermal activity from a left half of a body and a right half of a body.

15. (canceled)

16. The method of claim 14 wherein the capturing includes capturing electrodermal activity from a hand region of a body and a foot region of a body.

17. The method of claim 1 wherein the regions of time are based on one or more of physiological data and psychometric data.

18. The method of claim 1 further comprising determining deviations in electrodermal activity from a training session where no treatment is administered.

19. The method of claim 1 further comprising evaluating a treatment based on the electrodermal activity.

20. The method of claim 1 further comprising obtaining electrodermal activity for a plurality of people, performing signature analysis on the electrodermal activity to identify a characteristic of the electrodermal activity that corresponds to a pain, and evaluating a pain level based on the electrodermal activity for the plurality of people.

21. The method of claim 20 further comprising relating pain experienced by the plurality of people with the electrodermal activity for the plurality of people.

22. (canceled)

23. The method of claim 21 wherein the relating includes combining self-reported pain response with a pain stimulus start time and end time to define regions of pain responses.

24. The method of claim 21 wherein the relating includes comparing electrodermal activity during these regions for different types of pain stimuli to evaluate effects of a treatment.

25. The method of claim 20 further comprising deriving norms based on the electrodermal activity from the plurality of people and using the norms in evaluating a treatment.

26. The method of claim 1 further comprising validating the relating to distinguish a drug treatment from a placebo, including performing a statistical significance test.

27-28. (canceled)

29. The method of claim 1 further comprising obtaining a fundamental pattern present in the electrodermal activity from left and right body regions for each individual from a plurality of people and performing signature analysis on the fundamental pattern.

30. A computer program product embodied in a non-transitory computer readable medium for analyzing physiology, the computer program product comprising:
code for obtaining electrodermal activity for an individual; and
code for evaluating a pain level based on the electrodermal activity for the individual wherein the evaluating covers a plurality of regions of time and relates the electrodermal activity to self-report data for the individual.

31. A computer system for physiology analysis comprising:
a memory which stores instructions;
one or more processors coupled to the memory wherein the one or more processors, when executing the instructions which are stored, are configured to:
obtain electrodermal activity for an individual; and
evaluate a pain level based on the electrodermal activity for the individual
wherein evaluation covers a plurality of regions of time and relate the electrodermal activity to self-report data for the individual.

32-34. (canceled)
